(19) United States
(12) Patent Application Publication
(10) Pub. No.: US 2014/0017718 A1
(43) Pub. Date: Jan. 16, 2014

(54) ERYTHROCYTE MECHANICAL FRAGILITY TEST

(71) Applicant: Blaze Medical Devices, LLC, Ann Arbor, MI (US)

(72) Inventors: Michael Tarasev, Pinckney, MI (US); Kenneth Alfano, Canton, MI (US); Sumita Chakraborty, Ann Arbor, MI (US)

(73) Assignee: Blaze Medical Devices, LLC, Ann Arbor, MI (US)

(21) Appl. No.: 14/030,886

(22) Filed: Sep. 18, 2013

Related U.S. Application Data
(63) Continuation of application No. 13/708,980, filed on Dec. 8, 2012.
Application No. 13/708,980 is a continuation of application No. 13/213,576, filed on Aug. 19, 2011.
Application No. 13/708,980 is a continuation-in-part of application No. 13/213,576, filed on Aug. 19, 2011.
Application No. 13/213,576 is a continuation-in-part of application No. 13/412,691, filed on Mar. 6, 2012, now abandoned.
Application No. 13/213,576 is a continuation-in-part of application No. 12/690,916, filed on Jan. 20, 2010, now Pat. No. 8,026,102.
Application No. 13/412,691 is a continuation of application No. 12/690,916, filed on Jan. 20, 2010, now Pat. No. 8,026,102.
(60) Provisional application No. 61/146,145, filed on Jan. 21, 2009.

(51) Int. Cl. G01N 33/49

(52) U.S. Cl. CPC G01N 33/49
USPC 435/29; 435/288.7

(57) ABSTRACT

A test for erythrocyte membrane mechanical fragility, involving subjection of a sample comprising red blood cells to a mechanical stress capable of causing hemolysis, and measurement of the hemolysis present after particular extent(s) of the stress. Measurement of hemolysis can be without component separation, such as by cell-counting (optical or non-optical) or by spectral differentiation between cell-free and intracellular hemoglobin. Such tests can incorporate a disposable single-use component that contains an RBC sample during testing, and for certain embodiments the mechanical stress involved comprises (higher-energy) ultrasonic stress.

### CROSS-REFERENCE TO RELATED APPLICATIONS

This application is a US continuation application that claims the benefit of both Ser. No. 13/213,576, filed Aug. 19, 2011, and Ser. No. 13/708,980, filed Dec. 8, 2012. The Ser. No. 13/213,576 application is a continuation-in-part application of U.S. nonprovisional application Ser. No. 12/690,916, filed Jan. 20, 2010, which has since issued as U.S. Pat. No. 8,026,102 on Sep. 27, 2011, and which in turn claims the benefit of U.S. provisional application 61/146,145, filed Jan. 21, 2009. The Ser. No. 13/708,980 application is a continuation-in-part application of both Ser. No. 13/412,691, filed Mar. 6, 2012, and Ser. No. 13/213,576, filed Aug. 19, 2011, which are respectively a (now abandoned) US continuation application and the aforementioned US continuation-in-part application of the aforementioned U.S. nonprovisional application Ser. No. 12/690,916, filed Jan. 20, 2010, which has since issued as U.S. Pat. No. 8,026,102 on Sep. 27, 2011, and which in turn claims the benefit of U.S. provisional application 61/146,145, filed Jan. 21, 2009. All of these are herein incorporated by reference in their entireties for all purposes, and any claim(s) of this application may be based on matter from any one or more of such disclosures.

### FIELD OF THE INVENTION

This disclosure is in the field of medical and scientific methods and associated systems. In particular, it relates to fragility-measuring tests for red blood cells, and more particularly such tests employing mechanical stress.

### BACKGROUND OF THE INVENTION

This section contains general background material, which is not necessarily prior art.

Red blood cell (RBC, erythrocyte) membrane fragility can be measured in various ways, principally either osmotically or mechanically. In general, it involves subjecting (wholly or partially) a sample of cells to a stress/force and measuring how much hemolysis occurs as a result of the applied stress/force. In the case of mechanical fragility (MF), cell membranes are exposed to some kind of mechanical disturbance such as a shear stress—which may vary in intensity, duration, or other parameters—while the proportion of cells lysing is tracked. This enables cells' overall susceptibility to hemolysis to be characterized and presented (comprehensively or selectively) in various ways. Such susceptibility to lysis of cells can be referred to in other ways (e.g. stress-resistance or lack thereof, stability under stress, stress-susceptibility, hemolytic propensity under stress, etc.) or even indirectly (e.g. stress-caused hemolysis, stress-induced hemolysis, stress hemolysis, etc). Fragility indices or profiles (single-parameter or multi-parameter) for erythrocytes may be desirable for research purposes or for clinical purposes. Applications may include blood product quality testing, diagnostics, clinical research, or basic research.

### BRIEF SUMMARY OF THE INVENTION

This section briefly and non-exhaustively summarizes the subject matter of this disclosure.

Devices and methods for measuring red blood cell (RBC) fragility can be useful for characterizing blood product or patient blood, in a wide variety of possible applications. Fragility measurements involve some means for applying known sources/amounts of a stress, as well as some means for determining the extent of hemolysis resulting from particular extent(s) of the stress. There are different ways this extent-of-hemolysis can be measured (in conjunction with various means of applying the stress), including various types of spectral analysis as well as cell-counting.

This disclosure and others of its patent family pertain to RBC mechanical fragility testing, including by utilizing an integrated system to automatably combine the stressing and detection steps, and a (preferably single-use) disposable component for holding blood samples, with said component capable of serving in effect as both a stressing chamber and a detection chamber—albeit optionally in different portions of said component. In some embodiments, no fluidic transfer is needed within the disposable component between stressing and detection of portions of a sample. Employing a disposable/consumable cartridge or chip or other such piece for housing each sample to be tested can enable convenient testing of discrete samples with minimal cleaning or risk of contamination—among other benefits. Such single-use components can be configured to subdivide a given sample into multiple (discrete/separate/step-wise or continuous-flow based) subsamples to facilitate concurrent or otherwise disparate stressing for a single- or multi-dimensional profile, and/or be configured to receive multiple samples from multiple respective sources to facilitate multiplex testing.

This disclosure also addresses utilizing sonication, to subject RBC to high-energy mechanical stress, as part of a particular approach to measuring RBC mechanical fragility. “Low-energy” mechanical fragility, such as that utilizing a typical bead mill, tends to more directly reflect erythrocyte membrane properties (e.g. related to its integrity), whereas “high-energy” mechanical fragility, such as that utilizing a sonicator, tends to more directly reflect hemoglobin viscosity and cell size/volume. (Note that both general kinds of fragility assays could be useful for different purposes, and potentially could be used in conjunction.)

The scope of the invention is defined by the claims, which are incorporated into this section by reference. A more complete understanding of the present disclosure (including other filings that are incorporated herein by reference) and associated embodiments will be afforded to those skilled in the art, as well as the realization of additional advantages thereof, in consideration of the overall disclosure including drawings where applicable. s. Reference will be made to the appended sheets of drawings that will first be described briefly.

### BRIEF DESCRIPTION OF THE DRAWINGS

This section briefly describes the accompanying drawings for this disclosure. All drawings are for selectively and non-exhaustively illustrative and explanatory purposes only.

FIG. 1 shows a single-sample disposable cartridge for testing RBC fragility via sonication, the cartridge being in effect both a stressing chamber and an optical cuvette.

FIG. 2 shows a single disposable sample cartridge being inserted into an ultrasonic RBC fragility test system wherein a sonication head is ready to be moved into place.

FIG. 3 shows beneath the device shroud of an ultrasonic RBC fragility test system, wherein a sonication mechanism, cooling and alignment fixture, light emitter and optical detector, and control board are visible.

FIG. 4 shows a complete ultrasonic RBC fragility test system, with supporting computer (for control and analysis). Note that this particular version holds only one cartridge at a time, and each cartridge holds only one sample (although multiple such systems could be run by the same computer concurrently)

FIG. 5 shows a “multi-lane” ultrasonic RBC fragility testing system, which holds multiple single-sample cartridges for testing.

FIG. 6 shows a multi-sample disposable cartridge for testing RBC fragility via sonication, each section of the cartridge being in effect both a stressing chamber and an optical cuvette for its respective sample.

FIG. 7 shows a single-sample disposable cartridge for testing RBC fragility via sonication, wherein the sample gets split into multiple sub-samples, each section of the cartridge being in effect both a stressing chamber and an optical cuvette for its respective sub-sample.

FIG. 8 shows a single-sample, multi-sub-sample disposable cartridge placed within a casing containing components for cooling, sonication, and optical detection.

FIG. 9 shows a tower stacking multiple single-sample, multi-sub-sample disposable cartridges as “drawers,” each such drawer containing components for cooling, sonication, and optical detection of its respective sample, so as to integrate ultrasonic RBC fragility testing of multiple samples and their respective subsamples.

FIG. 10 shows a single-sample disposable cartridge to contain sample content during concentric-cylinder based testing of RBC mechanical fragility, the cartridge comprising both a stressing chamber and an optical cuvette.

FIG. 11 shows a core view of a base unit of a concentric-cylinder based system for testing of RBC mechanical fragility that utilizes a disposable cartridge to contain sample content during testing, the base unit comprising both a stressing portion and an optical portion, and with a door closed over the optical portion.

FIG. 12 shows a see-through view of a concentric-cylinder based RBC mechanical fragility test system from the front, which incorporates a disposable cartridge to contain sample content during testing.

FIG. 13 shows a see-through view of a concentric-cylinder based RBC mechanical fragility test system from the top, which incorporates a disposable cartridge to contain sample content during testing.

FIG. 14 shows a close-up front view of the optical portion of a concentric-cylinder based mechanical fragility test system for red blood cells.

### DETAILED DESCRIPTION OF THE INVENTION

This section contains descriptive content for this disclosure. It is also to be understood that the terminology and phraseology used herein is for explanatory and exemplary purposes and not intended to be limiting. This section may comprise paraphrase, abridgement, recapitulation and/or elaboration of select matter from earlier related (e.g. parent, grand-parent, etc.) disclosure(s). Some matter is incorporated by reference, as indicated, from indicated US patents and US published patent applications.

Erythrocyte mechanical fragility testing combines controlled physical (and more specifically, mechanical—e.g. shear, pressure, stretch) stressing of cells, along with a measurement of how much hemolysis occurs during such stressing. Most expressed definitions pertain to a notion of propensity for or susceptibility to hemolysis under mechanical stress. A wide range of research and clinical applications are possible for such metrics. Other (i.e., “non-mechanical”) kinds of fragility generally focus on other physical stresses—such as osmotic or photonic stress. Mechanical fragility has the potential to more relevantly (for select purposes) reflect erythrocyte membrane fragility and membrane integrity, since osmotic fragility for example significantly depends on non-mechanical aspects such as intracellular ion concentration and transmembrane water/ion rates as well as cell size (in addition to the mechanical properties). This is likely part of why osmotic fragility has not been usefully correlated to the viability of stored RBC, for example. (It is notable that even such “non-mechanical” types of fragility may be made more useful for blood-banking or other applications that they would otherwise be, thanks to the multi-dimensional approaches provided in the original parent application—as well as the possibility of combining stress types to expand the domain of investigatable correlations.) Sometimes the nomenclature or classification of stress types and related concepts can vary, especially at the boundaries or in cases of combinations or overlap. Likewise, within mechanical stress types, precise sub-categorization can vary as well (e.g. exact definitional limits or intersections of shear, pressure, stretch/tension, etc.). Note that “mechanical” fragility per se is generally understood as being distinct from “osmotic” fragility (although the two could also be combined), despite there being a mechanical effect on the cell wall as part of osmotic stressing. Likewise, references to “shear” stress/fragility presumptively refer to kind(s) of “mechanical” stress/fragility, unless some qualification and/or stress combination indicates otherwise.

There are many ways to provide the mechanical stress in a fragility assay, as well as many ways to measure the resulting hemolysis. As described by Tarssanen (1976), approaches to providing mechanical stress can be grouped into two broad categories: “high-energy” mechanical stress, and “low-energy” mechanical stress. Low-energy mechanical fragility, such as for example that utilizing a bead mill for the stress, tends to more directly reflect erythrocyte membrane properties, whereas high-energy mechanical fragility, such as that for example utilizing sonication for the stress, tends to more directly reflect hemoglobin viscosity and cell size. (Note that both general kinds of fragility assays could be useful for different purposes, and potentially for overlapping applications, and both kinds of stress are still “high” compared to that typically used for “deformability” measurements—in the sense of being potentially lethal to RBC and thus useful to hemolyze at least some of the cells as is needed for any “fragility” assay).

Certain kinds of mechanical stress, such as viscous stress, can readily be configured as high-energy or low-energy; also, a fragility profile for some stress mechanisms may reflect membrane properties more directly in the lower ranges but reflect surface-to-volume ratio and viscosity more directly in the higher ranges. The implications of these differing kinds of effects can be that fragility tests employing different stress means and/or energy levels can correlate differently to a given phenomenon; to some extent this can be true even among different forms of low-energy stresses (e.g., bead mill vs. capillary tube). Just as osmotic and mechanical fragility can sometimes have inverse correlations to certain phenomena, likewise so can high-energy and low-energy variations of mechanical fragility. Also, sometimes stressors can be combined, such as when evaluating changes in mechanical fragility under differing osmolarities; relatedly, temperature or pressure or other factors can in some cases be relevant environmental conditions—whether varied or not.

However the stress gets provided for a given fragility test, there are also many ways to in effect measure the hemolysis resulting from the stress. Whenever the particular advantages of cell-counting (an approach described in US patent application publication US20110300574 A1, published Dec. 8, 2011, which is incorporated herein by reference in its entirety for all purposes) are not needed, the preferred approach is to use the Blood Hemolysis Analyzer disclosed in U.S. Pat. No. 7,790,464, issued Sep. 7, 2010, incorporated herein by reference in its entirety for all purposes, which notably avoids the need for any separation steps (such as for example by centrifugation). In general, among all possible means for detecting/measuring hemolysis level/fraction (whether implicitly or explicitly), optical ones are preferred; among optical means, spectral ones are preferred; among spectral means, those utilizing absorbance are preferred. As determining fractional hemolysis requires knowing the original (pre-lysis) hematocrit or a reasonable proxy therefor such as total hemoglobin concentration, two possible approaches for obtaining this piece of information are as follows: 1) achieving 100% hemolysis in at least one subsample as part of the stressing step, and/or 2) employing additionally the spectral analysis of blood in the visible spectral range (which can provide total Hb, which is the combined intra-cellular and extra-cellular hemoglobin).

A fragility assay can involve outputting various kinds or amounts of fragility data—including specific values or indices, single-variable-parameter fragility profiles, and/or multi-variable-parameter (“multi-dimensional”) fragility profiles. (Note that terms such as “parameter” can be used to refer to either a stress parameter—which can be varied to create a profile—or a fragility index value which may itself be based on or derived from such a profile, depending on the context.) Data matrices comprising how much lysis occurs under various combinations of stress parameters can be used to yield profile-based parameters characterizing the sample tested (or the source it represents). Data of interest (or inferable information therefrom) could comprise how much lysis would be expected under a given set of stress parameters, what stress condition(s) would be expected to result in a given lysis level, or slopes or shapes of any such curves/trajectories—the latter of which can reveal subpopulations within a sample with their own discernible profiles. The distribution of cell ages (i.e., physiological/metabolic ages, distinguished from blood product/unit ages) within a given person or animal may be a factor in sub-populations within a sample exhibiting distinct charcteristics; nevertheless, often it is desirable for a single value (e.g., an average via some fragility-based metric) to represent an overall sample. The particular fragility parameter(s) sought will likely depend on what is deemed most clinically or scientifically relevant for each particular application. Notably, a single-value index parameter can itself be determined from a profile, such as for example through an interpolation (e.g., to estimate from available data points how much stress duration at a given intensity of a given type would be required to lyse a given percent of RBC in a given sample/source). Such fragility parameter(s) can be computed using a processor, which could be any kind of computer or like unit capable of generating desired output from raw measurements. Data points on a fragility profile may refer to cumulative induced hemolysis (i.e. not counting auto-lysis) or total overall hemolysis (i.e., including auto-lysis), among other conceivable presentations as desired for a given application. For simplicity, some versions may simply stipulate that accuracy of results could be compromised by significant auto-lysis in the sample (particularly if plotting as total lysis rather than induced lysis). Clinical versions may also incorporate a statistically validated “flag” for excessive auto-lysis (prior to performing the full fragility test).

Depending on the stress/lysis method employed for a given mechanical fragility (MF) testing approach, it may be important to dilute samples to ensure that all samples (or subsamples) have the same concentration of red cells (hematocrit) in order to have consistency in the rate/efficiency of hemolysis when subjected to stress. Later herein it is addressed the extent to which this is an issue for a sonication-based stress/lysis method. Also, in the case of red cell samples experiencing aggregation or coagulation, it can be useful to ascertain the role of such on the cells' susceptibility to induced hemolysis.

Sensitivity with regard to spectrally-based measures of hemolysis can be enhanced by accounting for multiple forms of hemoglobin—namely oxy, deoxy, meth, and/or carboxy. It's uncommon to determine the concentration of all four types, and perhaps unnecessary for the amount of precision typically needed (including for the present invention), but nevertheless remains an option. Other absorbent proteins that may potentially interfere with hemoglobin measurements can be accounted for with multi-wavelength analysis.

Clinical applications of fragility-based red cell metrics can include blood product quality testing by measuring sub-lethal cell damage (e.g., extent, rate, acceleration, trajectory, etc.) existing during storage any time after donation due to aging, processing, storage conditions, etc. Alternatively, fragility (osmotic and/or mechanical) can be useful in various patient diagnostics, including for example diagnosing conditions caused wholly or partially by drug and/or device based medical therapies—as well as any number of pathological diseases known or found to have an effect upon red cell membrane properties.

Regarding the “blood-banking/transfusion-medicine” (herein abbreviated “TM”) related uses, the related original application Ser. No. 12/690,916 (publication no. US 20100184120 A1, published Jul. 22, 2010, and incorporated herein by reference in its entirety) described previously-unexplored approaches for using in vitro testing of RBC membrane properties to reflect blood quality loss in a time-independent manner; in particular, it suggested using RBC fragility, preferably mechanical, and preferably via profiling, to indicate stored blood quality or loss thereof, and it notably presented preliminary in vitro data showing that stored RBC units of the same age can differ substantially in their mechanical fragility profiles (e.g., see FIGS. 6 & 7 and paragraphs 110-113 of that published application). U.S. Pat. No. 8,263,408, issued Sep. 11, 2012 (resulting from a related divisional application of the noted parent) is also herein incorporated by reference and it addresses “red blood cell suitability for transfusion,” tests for which can involve either a donor's (i.e. directly drawn from a donor or prospective donor) or donated (i.e. stored) red cells. Mechanical fragility (MF), especially at a relatively lower energy and/or intensity, is believed by the inventors to be superior to osmotic or other kinds of fragility for RBC quality-assessment purposes, due to its greater focus on membrane characteristics and its greater resemblance to stresses encountered by RBC in vivo (e.g. shear); moreover, “profiling” is believed superior to single-point MF metrics, due to its potential to provide a range of data from which select index(es) can be inferred/derived according to what provides the strongest or most clinically relevant correlations. RBC MF is also believed superior and more clinically relevant overall to all or most feasible measures of “deformability” (direct or indirect) for such quality-assessment purposes, because of MF's potential to reflect repeated or sustained stressing and to test cells to the point of intentionally breaching their integrity (testing to failure), as well as MF's high conduciveness to “multidimensional” profiling—also a comparative advantage of MF over osmotic fragility (OF). However, all such superiorities remain to be clinically proven, as thus far no in vitro test or metric based thereon has yet been validated as clinically superior to storage time or ST (hence why ST remains in effect the main indicator of pRBC quality or loss/degradation thereof). MF's potential to strongly reflect RBC membrane properties makes it appealing as a possible “aggregate” parameter or metric, as various other in vitro tests look at things (e.g. biochemical levels like ATP) whose ultimate cell-quality impact can in effect be substantially reflected collectively via MF. MF testing with RBC that are not from a donated blood product unit or donor thereof, in particular RBC of a patient's blood (whether being auto-transfused or remaining in the patient), is discussed later herein—in the context of blood-handling devices. Note that donated RBC samples may also be provided by patients or research subjects, for studying/researching any patient-diagnostic potentials of MF.

An extension of the blood product quality application can be to evaluate effects of blood product collection manner (e.g. apheresis vs. whole-blood) or manufacturing processes (e.g. leukoreduction, irradiation); or to validate associated processing equipment, as well as any storage/handling/transportation materials or conditions (e.g. bag material, storage or rejuvenation solutions, temperature ranges), by examining their respective effects upon blood quality or transfusion suitability (in terms of RBC fragility). Such RBC membrane properties can also be used to ascertain beforehand which units may be most amenable to certain manufacturing processes or storage conditions, by establishing a predictive correlation (directly or indirectly) between the in vitro property before subjection to said process or condition and relative in vivo performance. As with other applications, particular fragility assays or parameters may prove useful in combination with each other and/or other assays (e.g. biochemical).

Ongoing research in TM will progressively strengthen the case for clinical adoption of the MF test, with each round of studies likely enticing additional users for more use contexts (clinical opinion varies as to which kinds of studies would be most conclusive, as well as which applications would be most useful). Measurable degrees of quality/suitability could be used to reflect degrees of product acceptability, such as by selectively triaging those units deemed to be the “best” or least-degraded toward the most vulnerable patients (or for those unit transfusions where oxygenation is deemed the most critical, etc.). Moreover, the multi-parametric/multi-dimensional potential of MF could enable different “kinds” of quality to be discerned which may respectively prove more or less clinically relevant for different patients and/or patient groups/conditions. For example, post-transfusion in vivo RBC survival and RBC perfusion may each tend to be more associated with distinct respective multi-dimensional profile characteristics, as may resultant tissue oxygenation. Particular profile-based fragility parameter(s) or index(es) may thus prove indicative of RBC ability to adapt to the physical and/or chemical environment within the patient/recipient in a way that varies inter-donor, intra-donor, and even intra-unit (e.g., via sub-populations or cell-by-cell differences).

In effect, storage time has generally been the principal indicator of blood quality loss as applicable to particular individual units—although notably, the usefulness of even this indicator remains the subject of much controversy. This highlights the value of establishing a unit-specific RBC quality test to replace or supplement mere storage time as an indicator of storage lesion—as well as rates thereof, by testing at multiple points in time—thereby allowing modifications in today's inventory management practices (presently dominated by “first-in-first-out,” or FIFO). And in some cases, the RBC membrane properties of a patient's own blood may also be a factor in what kind of RBC properties are needed from stored blood they would receive in a transfusion. Variations among RBC units right at donation/collection can be at least partly attributable to “donor-to-donor” differences, which can also be reflected in subsequent degradation during storage (along with other factors). Dern et al. had first showed inter-donor variation in RBC “storageability” as measured by post-transfusion survival in vivo, and looked at some possible in vitro metrics (both membrane-related and strictly biochemical); Card et al. subsequently showed related findings (regarding inter-donor differences in certain RBC membrane properties). In some cases such differences may result from some identifiable medical condition, but there can also be a substantial range of variation among “healthy” donors. Also notable is that even “same-donor” differences can exist from one donation to the next.

In other (i.e., non-TM) applications outside the context of blood banking for transfusion, mechanical fragility (MF) can be used to evaluate or validate various blood-handling devices (or medical procedures employing them) by measuring sub-lethal RBC injury they cause manifesting in higher MF (e.g. Kamaneva et al. 2002, Yazer et al. 2008); relatedly, MF testing can be used to calibrate or account for MF to facilitate a consistent standard of evaluation of the actual hemolysis caused by blood-handling devices (e.g. Gu et al. 2005). Note also that evaluation of blood-handling devices via their effects on patients can be distinguished from actual patient diagnosis of a condition for which such a device may in fact be a cause—and MF could be useful for either or both of these.

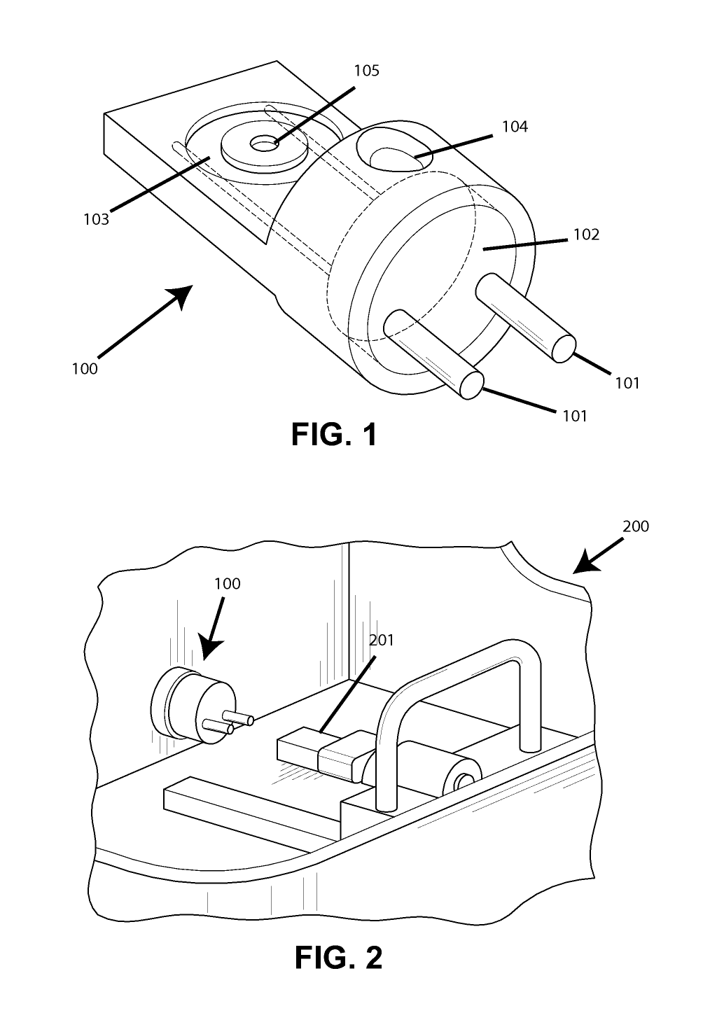

There still as yet is no well-established or universally-standardized means to test for RBC MF—a fact that may have limited its utility for any of the above-noted or other applications. And likewise, the non-integrated and manual-labor-intensive nature of previous MF testing methods and means pose a challenge to such a test achieving such establishment or standardization. However, an integrated approach or method (preferably automated, particularly regarding any transfer of sample between stress subjection and lysis evaluation, unless both occur at the same place as in the sonication-based example of FIGS. 1-9) utilizing a system such as for example the embodiment depicted in FIGS. 10-14 (whereby FIG. 12 shows an overall system, and FIG. 10 shows a cartridge) can help facilitate greater use of MF testing. Of course, a vital aspect of optimal implementation for any application of any new life-science test includes the accumulation of copious output and associated correlations, so that with time the relevant characterizations become progressively more meaningful and accurate. Likewise, hurdles to broad general acceptance of any new clinical test tend to require more conclusive evidence than is needed to begin piloting select applications among early adopters (or to use for research purposes). Furthermore, successful clinical validation may involve targeted investigating of correlations specifically exhibited with particular patient groups or conditions.

Clinical feasibility of MF testing for any diagnostic or other applications is facilitated by utilizing devices or systems capable of combining, in a single integrated system, stress application with measurement of induced hemolysis (as well as with results processing and output) for adequate speed, reliability, operability, etc. Moreover—for research uses and especially for clinical uses of MF testing—convenience, safety, and reliability can be substantially enhanced by also employing a disposable (ideally single-use) chip or cartridge component or the like to self-contain the blood sample during testing. A disposable component can allow several advantages for many of the above-noted applications of MF testing, depending upon the overall MF testing approach and configuration; usage of a replaceable disposable component for holding one or more blood samples to be tested during any given operation of the system can be more conducive to designing for concurrent or successive replication, for multiplexing either the dimensionality of fragility profiles and/or the number of distinct samples being tested at once while protecting against cross-contamination. It may also be more conducive to incorporating system calibration capabilities specific to a given configuration and/or kind of sample (e.g. pRBC), to further improve the reproducibility and reliability of test results. And notably, the integration of the stressing/lysing function with the detection/evaluation function helps to facilitate incorporation of such a disposable piece.

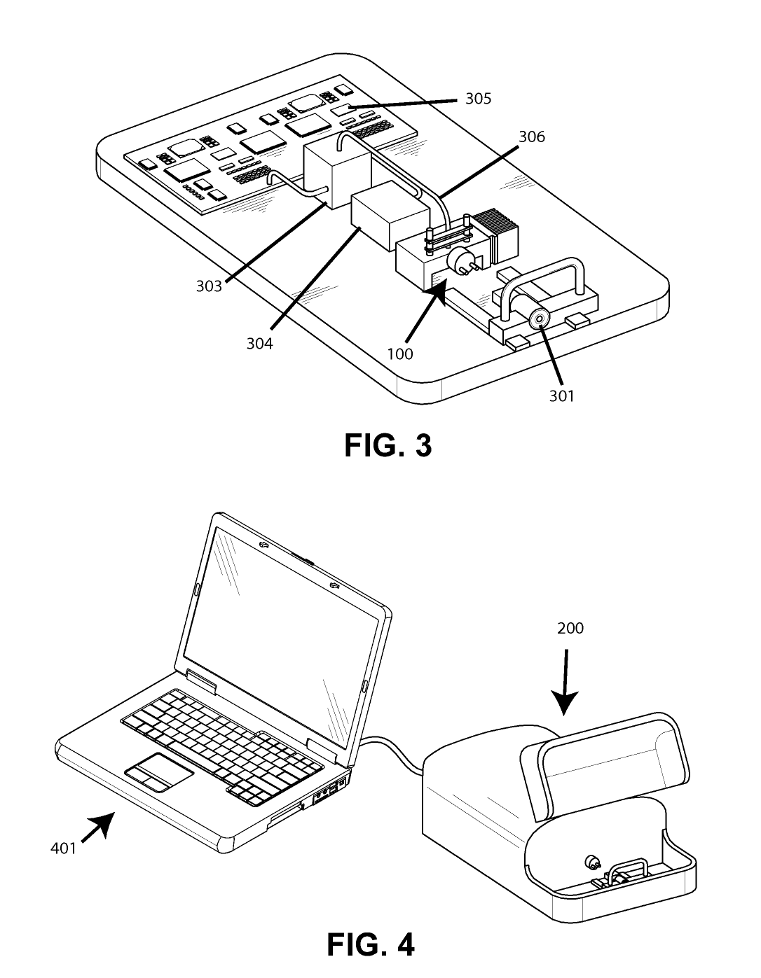

Various approaches for stressing and/or detection can be approached in conjunction with employing a single-use disposable component for containing the sample during the stressing (and preferably during the detection as well, albeit optionally in a different part thereof). In the context of a system directed toward blood product quality testing, for example, a rather generalized form of the disposable-utilizing approach is in claim 8 and in FIGS. 3 and 4 of U.S. Pat. No. 8,268,244, issued Sep. 18, 2012, which is herein incorporated by reference in its entirety, and which was a divisional of the original '916 application. Although that patent was primarily focused upon blood-banking/transfusion applications of fragility testing, that example (comprising a concentric-cylinder based stressor unit with optical detection for measuring induced hemolysis) is more generally-applicable as well for providing automated integration of MF testing (as also noted above, referring particularly to FIGS. 10 and 12 herein, which overview the example embodiment depicted by FIGS. 10-14).

Ultrasound (also US, or U-S) via sonication is another one of the possible approaches for providing some or all of the mechanical stress in a MF assay. U-S involves multiple causes of its resulting hemolysis, which can be induced via fluidically-translated mechanical forces (a physical cause) and also via free radicals generated in the sample (a non-physical cause), the latter of which can be a confounding factor that is generally best to minimize in a fragility test so that “physical” causes will predominate.

For an U-S based fragility measurement to be accurate, the application of physical stress needs to be configured to ensure that other (non-physical) potential causes of hemolysis are not unduly affecting the results—which are supposed to primarily reflect susceptibility to applied physical stress, at least for uses where non-physical effects are not desired to be reflected in fragility results. In such cases, hemolysis from radicals should ideally be essentially negligible in comparison to hemolysis from physical effects. Hence, to usefully employ sonication for a fragility assay, it is important to ascertain the relative extent to which radicalization is responsible for the hemolysis. Existing literature indicates that generally the physical effects predominate over the chemical (radical) effects. Preliminary experiments conducted by the present inventors largely agree with this, and further indicate that this is mainly true for relatively low ultrasound intensities—the particular values for which vary of course by the particular configurations employed. These experiments were conducted with a bench-top sonicator and they compared RBC units with and without reagent for negating the effect of radicalization, in order to assess the relative roles of physical and chemical causes for ultrasound-induced hemolysis at different ultrasound intensities (power levels). Aside from power intensity and duration, other variable stress parameters could include for example sonication frequency as well as various solvent/solution properties. The maximum desirable intensity threshold may need to be experimentally-determined for each geometry, volume, etc., and depend on whether direct or indirect contact is employed. (Note that this issue of U-S “intensity” as discussed here is not necessarily the same as the earlier-discussed issue of high-energy versus low-energy types of stress, though they could be related concepts in principle; some data has shown that lower intensity levels of the lower-energy kinds of stress can better showcase differences between samples w.r.t. membrane integrity.)

Regarding the matter of cell concentration with a U-S based fragility test, existing literature establishes the general principle that cell concentration (hematocrit) is a factor for hemolysis efficiency. The inventors’ preliminary experimentation largely agrees with this, and further indicated (through serial dilution experiments performed with a bench-top sonicator) that when the hematocrit is low enough (for a given ultrasonic stress intensity) this can essentially be neglected. (The particular threshold levels for dilution and associated sonication intensity may need to be determined experimentally for any given configuration.) Hence, either dilution should be normalized to establish a uniform hematocrit, or else dilution should be high enough to avoid the need for normalization.

Various other aspects of a US-based MF testing parameters tend to need to be empirically validated and optimized, some of which are configuration-specific and some of which might be fairly generalizable. For example, inventors' preliminary data suggests that “pulsing” vs. continuous application of ultrasound does not seem to make an appreciable difference so long as the cumulative duration of ultrasound application is the same and the temperature increase due to US power dissipating in the sonicated sample is ensured to not be significantly affecting the sample's properties (e.g. sample viscosity or RBC membrane fluidity). On the other hand, for example, sample volume is a significant factor in lysis efficiency and thus must always be accounted for.

Unlike with some other mechanical stressors, U-S stress does not necessarily involve the sample interacting principally with a solid object or rigid surface—a fact that makes sonication somewhat more conducive to occurring in the same chamber as detection. Yet this can optionally be changed, such as if combined with commercially-available micro-beads or the like, which may introduce a lower-energy bead-induced stress in combination with the higher-energy U-S stress. Note that if combining multiple different means or sources of stress together, their respective contributions to any results must be studied in order to assess their combined suitability for any given application. More generally, another benefit of employing disposable cartridges for the samples is the ability to provide different kinds of cartridges compatible with a given system—thereby allowing a change in the assays being run merely by changing the kind of cartridge being used. For example, some cartridges may be selectively offered with built-in micro-beads of varying sorts, different buffer solutions or internal dimensionalities, etc.

In an alternative set of embodiments of a related device or corresponding method, a sample can be split into distinct subsamples which respectively receive only a high-energy mechanical stress (for example, via a sonicator) and only a lower-energy mechanical stress (for example, via a conventional bead mill). Hemolysis can be tracked in each case to give a fragility profile for each respective kind of stress. Such joint profiling could then be used to produce a joint index whose value comprises some blend of the information obtained from the two profiles. As a simple example, one potential joint index could comprise an average of the durations of the two different stress kinds at given intensities that each corresponds to 50% hemolysis in their respective subsamples. This could be useful in cases where high-energy MF profiles and low-energy MF profiles may tend to differ substantially. For example, stored blood tends to experience increases in membrane rigidity as well as changes in shape; hence, this might be expected to result in degraded blood having a greater susceptibility to certain stressing means, while a lower susceptibility to others (which could be tracked over time during storage, by a joint index). Embodiments of this approach can optionally involve using consumable/disposable pieces also.

This disclosure now describes certain example approaches and embodiments for particular aspects of a MF tester (as depicted in the accompanying figures). It can be observed that for the sonication-based system embodiment (e.g. FIGS. 1-9), the need for manual transfer of sample is avoided by using the same chamber for both causing and measuring hemolysis; for the concentric-cylinder-based system embodiment (e.g. FIGS. 10-14), it is achieved by strategically employing a peristaltic pump which provides automated transfer of a sample within the cartridge/disposable piece (as described below). Note how both examples of MF test integration do employ substantial automation, but the former does not require fluid transfer (manual or automated) to occur between the stressing and detecting functions.

FIG. 1 shows a disposable sample-holding component 100, structured in this case as a replaceable cuvette/cartridge that can serve as a combined chamber for contents undergoing stress, in this instance via sonication (administered via probe 101 and transferred via gasket 102), and also for taking optical readings via its compressible/flexible region 103 (above and below which fiber optics can close in to take readings at desired cycle intervals), and that region can be pinched between stress intervals to achieve an adequate spectrophotometric gap. It would be filled with blood via the sample port 104 before its insertion into the device. The sonication probes are disposable, and built into the cuvette. The detection window 105 is mounted on a film layer which enables the device to accurately capture a thin (predetermined height) layer of blood within the detectable portion of the cuvette at each detection cycle.

As shown next in FIGS. 2, 3, and 4, the consumable fits into a bench-top unit, which contains the necessary interfacing electronics and mechanical fixtures, which in turn plugs into a computer for software control. Specifically, FIG. 2 shows a single-sample cuvette 100 inserting into the base unit (benchtop/tabletop) device 200, with the sonication head 201 ready to be moved into place before the lid of the device is closed. FIG. 3 shows the interior of the base unit, where beneath the device shroud is the sonication driver 301 aligned with the cartridge 100 which is held in an alignment and cooling (temperature stabilization) fixture 302. A light source 303 and an optical analysis unit 304 are controlled from the control/analysis board 305 and can connect optically to the cartridge via fiber optics 306. FIG. 4 then shows the overall system for testing a single non-split sample, with supporting computer 401 for control and analysis, which could have instead optionally been incorporated directly into the base unit 200 of the test system. (It is possible that multiple single-sample base units could be connected to one USB hub and run from the same computer, to allow processing of multiple samples at the same time.)

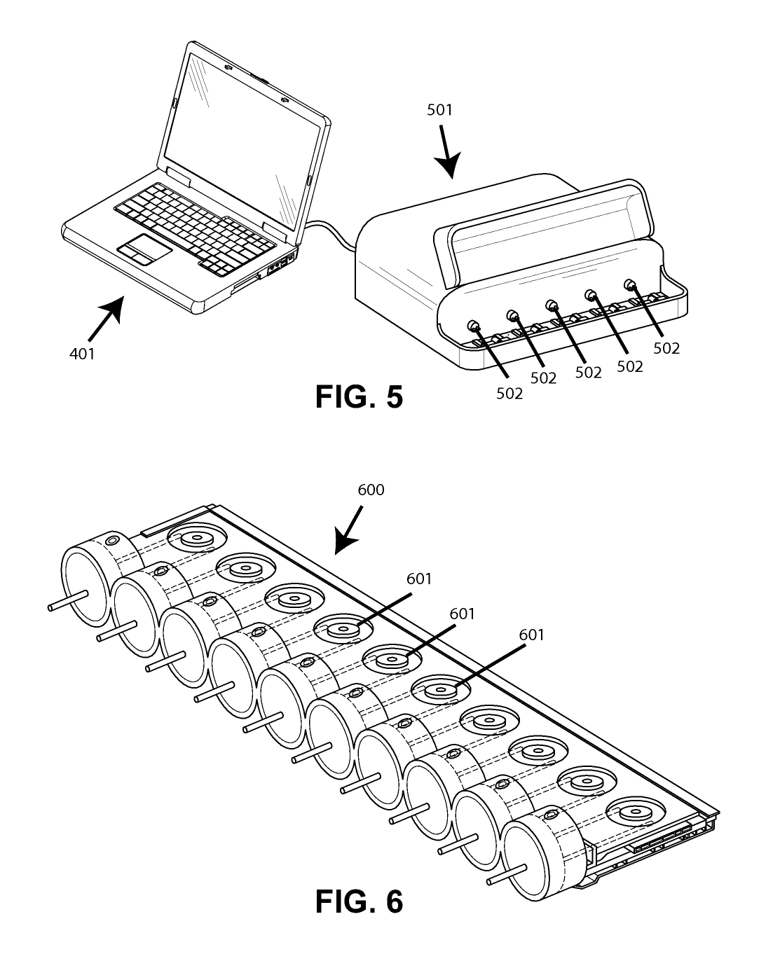

Alternatively, FIG. 5 shows how a multiplexed base unit 501 can accommodate multiple single-sample cartridges at once via parallel lanes 502. (This would be primarily a re-packaging of the single-lane base unit, as it would use the same single-lane cuvette for each blood sample or sub-sample to be tested.)

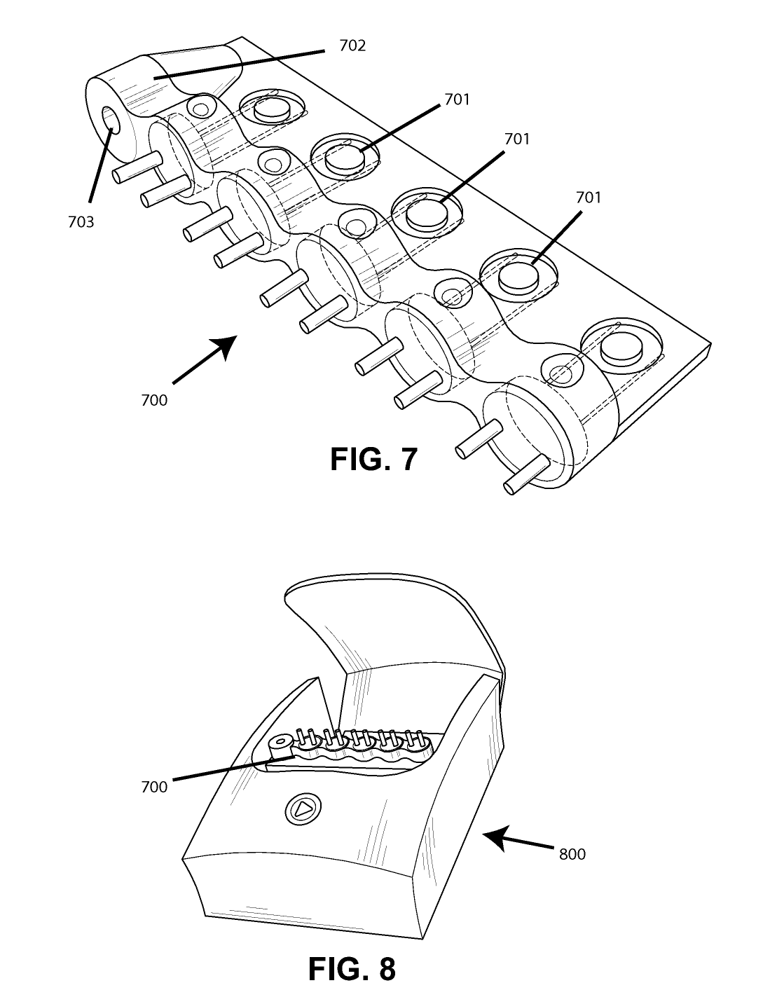

As further shown in FIGS. 6 and 7, the single chamber/cuvette concept from FIG. 1 can itself be replicated as a modular unit for multiplexing either the dimensionality of fragility profiles (e.g., via splitting a sample into subsamples going to multiple subsystems each providing a different stress intensity for a range of durations), and/or the kinds of fragility being tested (e.g., with and without beads), and/or the number of distinct samples being tested at once. Specifically, FIG. 6 depicts a multi-sample cartridge 600 consisting of linked cuvettes 601 for multiple distinct samples to be run simultaneously (each one as before), while FIG. 7 depicts a single-sample but multi-sub-sample cartridge 700 which aliquots a single blood sample into sub-samples (automatically) so that different stress/lysis conditions can be applied to each “sub-cuvette” or subsample-cuvette 701 to facilitate multidimensional fragility profiling of the sample. In FIG. 7, a common-receptacle 702 at one end takes a single sample through its port 703 and is linked to the bottom of each sub-cuvette such that the fluid level can equilibrate before insertion into the device. (Upon insertion, a membrane on the cartridge is sealed, trapping each sub-sample into its respective sub-cuvette and isolating it from the other sub-cuvettes.)

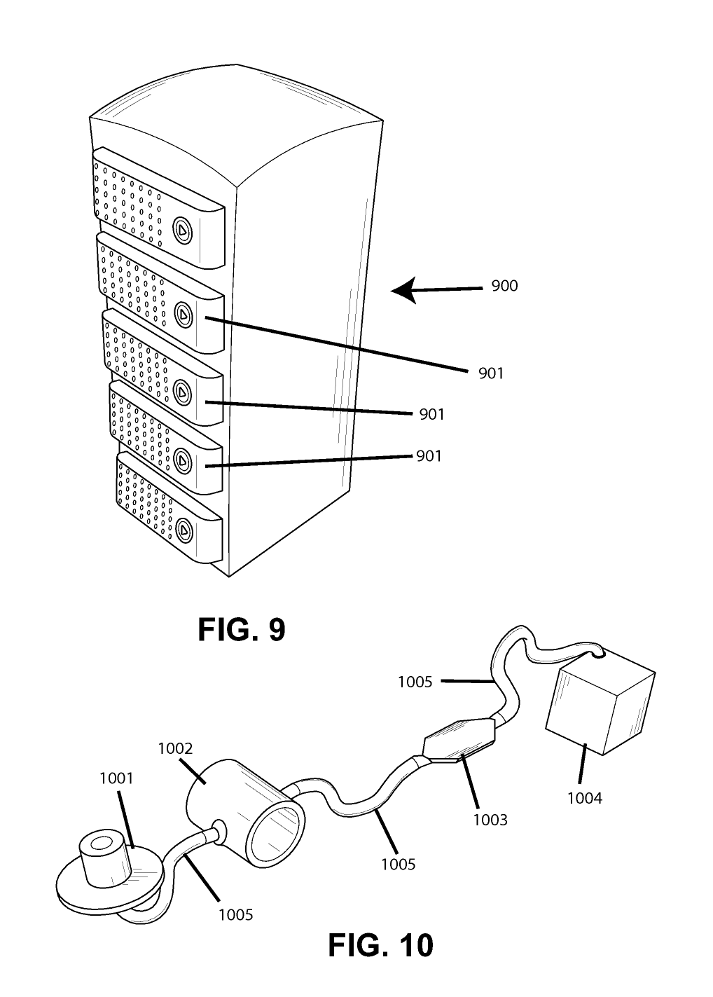

FIGS. 8 and 9 then depict a way to combine splitting a sample into subsamples (e.g. for multi-dimensional profiling of each sample) while also testing multiple different samples at once. Each distinct sample is given its own multi-compartment cartridge for splitting into subsamples, and placed in its own casing device, and then multiple such devices are then run in parallel by a single computer. As shown in FIG. 8, each single-sample/multi-sub-sample cartridge 700 can be inserted into a small casing device 800 containing the cooling, sonication, and optical features (not shown) for each sub-cuvette. FIG. 9 then shows a multi-cartridge tower 900 which would allow such cartridges in their respective casings to be inserted as tower “drawers” 901.

FIG. 10 shows an example of an alternate embodiment of the disposable cartridge (i.e., sample-holding component) having separate portions configured for contents undergoing stress (in this instance via concentric cylinders, with sample residing in the gap while one of them rotates) and optical detection (in this instance via a thin rigid cuvette portion, to which sample flows from the stressing portion). A syringe dock 1001 is connected to a lysis chamber 1002 via tubing 1005. The lysis chamber 1002 is connected to an optical cuvette 1003 via tubing 1005. The optical cuvette 1003 is connected to a waste reservoir 1004 via tubing 1005.

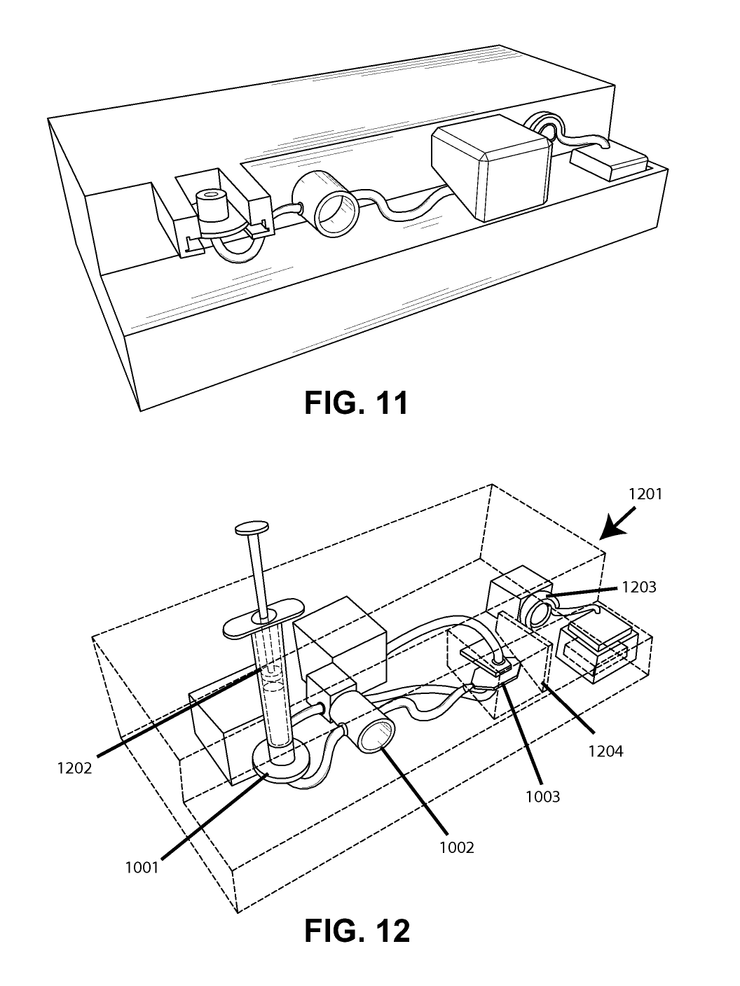

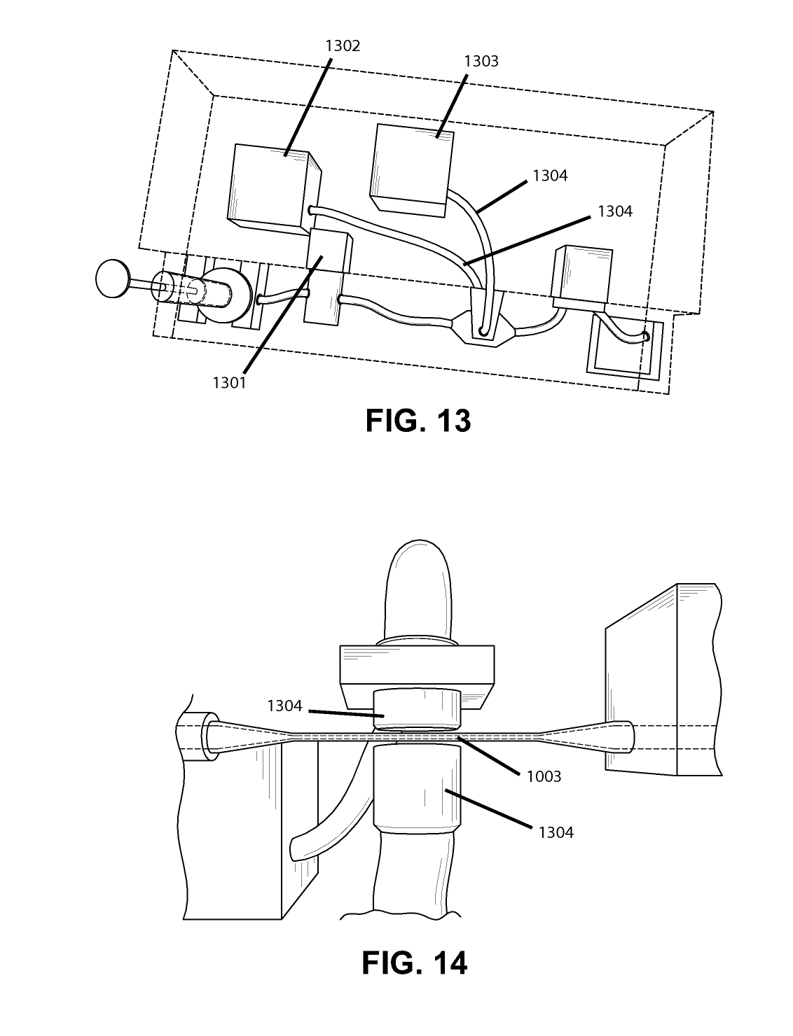

FIG. 11 shows a basic view of how the disposable from FIG. 10 fits into a corresponding base unit (benchtop/tabletop device) for a mechanical fragility test system, while FIGS. 12 and 13 show a see-through view of the device from the front and top, respectively, and with a door closed over the optical portion. A syringe 1202 injects a sample into the syringe dock 1001. An integrated peristaltic pump 1203 moves sample through the disposable component for processing by respective portions of the overall base unit 1201, such processing including cell lysis in the lysis chamber 1002 wherein an inner cylinder is turned by a built-in motor 1301, and also subsequent analysis of the sample in the optical cuvette portion 1003 occurring under a closed light-tight door 1204 via a spectrophotometer 1302 in conjunction with a light source 1303 which both employ fiber optic bundles 1304 to connect (optically) to the cuvette. FIG. 14 shows a frontal zoom of the optical detection portion, wherein said fiber optics 1304 sandwich said cuvette 1003.

Fixed or normalized dilution of cell concentrations can be performed either by the user or automated in the system. Alternatively, the consumable piece could be pre-filled with buffer from manufacture. Note that in the case of stored/packed RBC (pRBC), the storage solution for the main bag may be different from that of the test-segments (which could be a factor in whether such segments are deemed sufficiently representative of the main bag for a given purpose).

In an alternate embodiment applicable particularly for testing stored RBC in a bag, fragility measurement potentially could be performed without requiring a test sample being extracted. This could involve, for example, an optical hemolysis analysis component capable of directly measuring through bag material, which clamps or closes upon a portion of the bag which when in a clamped position contains only a small portion of the blood product (optionally, such cabined portions could remain sealed afterwards as well, depending upon the means used). In conjunction with this is also a stressor component, which subjects said small portion(s) of contents to stress while it remains in the bag; this could in effect make the bag itself a sort of a disposable container for any samples so cabined therein.

It's important to contrast any kind of RBC “fragility” with the related property of cell “deformability”—which is a broad concept covering many different kinds of tests that all in some way seek to determine how well a cell can deform or change shape under some kind of stress. Moreover, fragility (MF in particular) is particularly well suited for multi-parameter (>1) stressing to give multi-dimensional (>2) profiles showing how hemolysis depends on two or more stress variables such as extent/degree of intensity and extent/degree of duration (for one or more given type/kind) of mechanical stress. Indeed, merely providing the available option of such data richness can potentially enhance the general utility of embracing MF over other RBC membrane-related metrics.

A cartridge-based (or the like) system could also potentially be adapted to test mechanical fragility of material other than red blood cells. Of course the stressor(s)' selection and configuration would need to be empirically assessed and modified as appropriate to suit such alternative material, and for a type of cells or tissue other than red blood cells an appropriately modified spectral or cell-counting approach to detection of lysis/rupture would be needed.

This disclosure is enabling to those of ordinary skill in the art, while maintaining adequate flexibility for reasonable adaptation. Moreover, those skilled in the art will appreciate variations of the examples and principles described herein, which are also intended to be within the scope of the present invention. Any references herein to “the invention” or the like are thus intended in this spirit.

We claim:

1. A method for assessing quality of specific units of stored blood product, comprising:
subjecting a sample containing red blood cells from a specific unit of blood product to at least one kind of mechanical stress to at least one degree of said stress, thereby causing hemolysis of at least a portion of said cells, whereby said at least one kind of mechanical stress to at least one degree of said stress comprises one or more set(s) of stress condition(s);
evaluating how much hemolysis occurs under one or more of said set(s) of stress condition(s), thereby determining a fraction of cells that are hemolysed upon respective set(s) of stress condition(s); and
generating a representation of quality for said blood product, said representation of quality based at least partly on red blood cell mechanical fragility as reflected by said fraction;
wherein the subjecting and evaluating steps are both performed via an integrated system that does not require the manual transfer of said sample between said steps.

2. A multiparameter method for characterizing patient or stored blood, comprising:
subjecting a sample containing red blood cells to at least one kind of mechanical stress, thereby causing hemolysis of at least a portion of said cells, wherein said stress is applied at two or more degrees of stress intensity and two or more degrees of stress duration; and
evaluating how much hemolysis occurs in said sample under two or more degrees of stress intensity and two or more degrees of stress duration, thereby determining what fraction of cells is hemolysed under multiple respective sets of stress conditions, thereby enabling a representation of red blood cell mechanical fragility based on stress parameters comprising stress intensity and stress duration;
wherein the subjecting and evaluating steps are both performed via an integrated system that does not require the manual transfer of said sample between said steps.

3. A method for assessing prospective transfusion efficacy of red blood cells, comprising:
subjecting a sample containing donated or stored red blood cells to at least one kind of mechanical stress for one or more durations of said stress at one or more intensities, causing hemolysis of at least a portion of said cells;
evaluating said hemolysis to create one or more data point(s), wherein each data point corresponds to a fraction of cells that were hemolysed under a respective intensity and duration of said stress, so that said data point(s) reflect red blood cell mechanical fragility for said sample; and
generating a numerical value(s) representing red blood cell suitability for transfusion, said numerical value(s) being based on said one or more data point(s);
wherein the subjecting and evaluating steps are both performed via an integrated system that does not require the manual transfer of said sample between said steps.

4. The method of claim 3, wherein the method is performed on multiple samples representing multiple blood product units in an inventory thereof.

5. The method of claim 4, further comprising translating said value(s) corresponding to each of said units into determinations of which units are acceptable or preferable for particular recipients.

6. The method of claim 3, wherein said numerical value(s) is an index based on a fragility profile comprising two or more of said data point(s), said data points being distinct so that each data point corresponds to a level of cumulative induced or total hemolysis present after a distinguishable extent of said duration and/or said intensity.

7. The method of claim 3, wherein said suitability for transfusion reflects donor-to-donor variability, storage solution(s), modifying additive(s), bag material(s), or condition(s) relating to production, storage, or transport.

8. The method of claim 3, wherein said suitability for transfusion reflects a rate and/or acceleration of quality degradation in vitro.

9. The method of claim 3, wherein said suitability for transfusion reflects damage from blood manipulation during collection from a donor.

10. The method of claim 1 or 2 or 3, wherein said stress is provided via concentric cylinders, whereby a cylinder spins at a given speed(s) while said sample resides wholly or partially in a gap between said cylinders for a given time(s), and whereby any conditions of the cells' environment affecting hemolysis efficiency under the stress remain essentially constant, such that said speed(s) and said time(s) are essentially the sole determinants of said hemolysis.

11. The method of claim 1 or 2 or 3, wherein the evaluating step is accomplished by ascertaining a ratio of intracellular hemoglobin to cell-free hemoglobin in said sample or subsamples thereof, thereby reflecting said fraction, without requiring separation of liquid elements from solid elements.

12. The method of claim 1 or 2 or 3, wherein said sample wholly or partially resides in a disposable component during the subjecting and evaluating steps.

13. The method of claim 12, wherein said subjecting occurs via concentric cylinders in one portion of said component and said evaluating occurs via optical detection at another portion of said component.

14. The method of claim 1 or 2 or 3, wherein the evaluating step is accomplished by cell-counting to ascertain red blood cell concentration in said sample or subsamples thereof both before and after the subjecting step.

15. The method of claim 1 or 2 or 3, wherein said stress comprises sonication, and wherein said subjecting and said evaluating both occur in a single location, such that no manual or automatic transfer of said sample is required between said subjecting and said evaluating.

16. A method for measuring red blood cell mechanical fragility, comprising:
subjecting a sample containing red blood cells to at least one kind of mechanical stress to at least one degree of said stress, thereby causing hemolysis of at least a portion of said cells, wherein said at least one kind and/or said at least one degree comprise one or more set(s) of stress condition(s); and
evaluating how much hemolysis occurs under one or more set(s) of stress condition(s) comprising a given kind and/or degree of said stress, thereby determining a fraction of cells hemolysed upon respective set(s) of stress condition(s);
wherein the subjecting and evaluating steps are performed via an integrated system that does not require the manual transfer of said sample or portion(s) thereof between said steps.

17. The method of claim 16, wherein said degree of stress is varied by both intensity and duration during the subjecting.

18. The method of claim 16, further comprising producing an index based on a fragility profile, a fragility profile comprising two or more distinct data points that each correspond to a respective set of said stress condition(s).

19. The method of claim 16, wherein the red blood cells are not from donated or donor blood but are from a patient's blood, and wherein the stress is provided by a blood-handling device in assessing said device's impact upon said patient's blood.

20. The method of claim 16, wherein said stress is provided via concentric cylinders, and said sample is wholly or partially moved via peristalsis after the subjecting step, thereby allowing a continuous flow of red blood cells between the subjecting step and the evaluating step, thereby allowing finer gradations for degree of stress duration than is possible with discrete or separate subsamples.

21. The method of claim 16, wherein the evaluating step is accomplished by ascertaining a ratio of intracellular hemoglobin to cell-free hemoglobin in said sample or subsamples thereof, thereby reflecting said fraction, without requiring separation of liquid elements from solid elements.

22. The method of claim 16, wherein said sample wholly or partially resides in a disposable component during the subjecting and evaluating steps.

23. The method of claim 22, wherein the subjecting occurs in a first portion of said component and the evaluating occurs at a second portion of said component.

24. The method of claim 16, wherein the evaluating step is accomplished by cell-counting to ascertain red blood cell concentration in said sample or subsamples thereof both before and after the subjecting step, such that a spectral analysis is not needed for said evaluating step.

25. The method of claim 16, wherein the subjecting and evaluating both occur in a single chamber so that sample movement is not required in order for the evaluating to occur following the subjecting, and wherein said stress comprises sonication.

26. A system for performing the method of claim 16, comprising:
a subjecting unit for subjecting wholly or partly a sample containing red blood cells to at least one kind of mechanical stress to at least one degree of said stress, thereby causing hemolysis of at least a portion of said cells; and
an evaluating unit for evaluating how much hemolysis occurs under one or more set(s) of stress condition(s) comprising a given kind and/or degree of stress, thereby determining explicitly or implicitly a fraction of cells hemolysed upon respective set(s) of stress condition(s);
wherein the subjecting and evaluating can be performed without requiring the manual transfer of said sample or portion(s) thereof between the subjecting and evaluating.

27. The system of claim 26, wherein said degree of stress is variable by both intensity and duration during the subjecting.

28. The system of claim 26, further comprising a processor for producing an index based on a fragility profile.

29. The system of claim 26, wherein said evaluating unit comprises an optical detection unit, and wherein said optical detection unit comprises an optical cell counter.

30. The system of claim 26, wherein said subjecting unit comprises a sonicator probe(s).
